(19) United States
(12) Patent Application Publication
(10) Pub. No.: US 2016/0351078 A1
(43) Pub. Date: Dec. 1, 2016

(54) ULTRASOUND IMAGING SYSTEM WITH IMPROVED TRAINING MODES

(71) Applicant: FUJIFILM SonoSite, Inc., Bothell, WA (US)

(72) Inventors: Craig Chamberlain, Seattle, WA (US); Amanda Mander, Bainbridge Island, WA (US); Luke Baldwin, Lake Stevens, WA (US); Marco Daoura, Bothell, WA (US)

(21) Appl. No.: 14/725,670

(22) Filed: May 29, 2015

(51) Int. Cl. G09B 23/28; G09B 5/06; A61B 8/00

(52) U.S. Cl. CPC G09B 23/286; A61B 8/54; A61B 8/461; G09B 5/06

(57) ABSTRACT

An ultrasound imaging system includes a control with which a user can view one or more training materials regarding how to use the system or perform an examination. The training materials are associated with one or more of the operating parameters of the ultrasound system. Upon selecting a help me control, a search is performed for those training materials that are associated with one or more current operating parameters of the ultrasound system. In another aspect, training materials include a record of one or more operating parameters used or described in the content of the training materials. When viewing training material, a user can select a “show me” control on the ultrasound system, which causes the operating parameters used or described in the training material to be loaded into the circuitry of the ultrasound machine. The user can then operate the ultrasound imaging system with the same imaging parameters used or described in the training material being viewed.

### TECHNICAL FIELD

The disclosed technologies relate to ultrasound imaging systems and in particular to training systems for ultrasound imaging systems.

### BACKGROUND

While advances in technology have made ultrasound imaging machines easier to use, there is still a high degree of operator skill required to obtain quality images of various regions of interest in a patient's body. Factors such as the optimal machine settings and the way in which a probe is held and moved on the patient all can have an effect on the quality of the images produced.

To teach physicians and ultrasound technicians how to obtain the best images, imaging systems typically come with training manuals and video tutorials. The pupil is expected to study a manual and watch the training videos and then try to duplicate the examinations described. Such training materials are often viewed on a computer screen or other video monitor that is different than the display of the ultrasound machine being used. Therefore, the user has to keep notes of the suggested machine settings and manually configure the ultrasound machine with the same settings before attempting to practice a particular examination. Similarly, there are times when a user is using the ultrasound machine and has questions about how to adjust a particular setting in order to improve an image. In the past, the user had to look up the current imaging mode and region of interest being imaged in the training manual and then attempt to duplicate the recommended settings to improve an image. Both of these training solutions can be cumbersome and inefficient.

### SUMMARY

To improve on the systems described above, the disclosed technology relates to an ultrasound imaging machine with a built-in training system. A memory in the ultrasound machine stores one or more of training instructions, sample images and video/audio tutorials that explain and illustrate the best practices for operating the imaging machine and for imaging a particular region of interest. In one embodiment, each of these training materials is associated with one or more tag values that relate the subject of the training material to an imaging mode, a particular region of interest or one or more machine settings.

In addition or alternatively, an ultrasound machine includes a memory in which a record of one or more current imaging parameters is stored. A “help” control is available to the user as a key on a keyboard, a soft key on a user interface screen or via some other user input mechanism. If the help control is selected, a learning module executed by the processor searches for training materials with tag values that match one of more of the current imaging parameters. A list of training materials that match or are related to one or more of the current machine parameters is presented to the user. Upon selection of a particular item of training material, the selected training material is presented on one or more video displays that are used by the ultrasound machine.

In another embodiment, training materials include a record of one or more imaging parameters that are used or discussed in the training material. The ultrasound imaging system includes a “show me” control on a keyboard or as a soft key on a user interface or as some other input control. Upon selection of the show me control, one or more imaging parameters associated with a particular piece of training material that is being reviewed are loaded into the imaging circuitry of the ultrasound system so that the user can operate the system using the same machine settings used in the training material. The user can switch back and forth between capturing live images with the settings used in the training materials and viewing the training material.

### BRIEF DESCRIPTION OF THE DRAWINGS

FIG. 1 is a block diagram of an ultrasound imaging machine in accordance with one embodiment of the disclosed technology;

FIG. 2 is a flow chart of steps performed by a learning module to find training materials that are relevant to one or more current imaging parameters that are in use on the ultrasound imaging machine; and

FIG. 3 is a flow chart of steps performed by a learning module to load one or more imaging parameters associated with an item of training material that is being reviewed into the circuitry of the ultrasound imaging machine.

### DETAILED DESCRIPTION

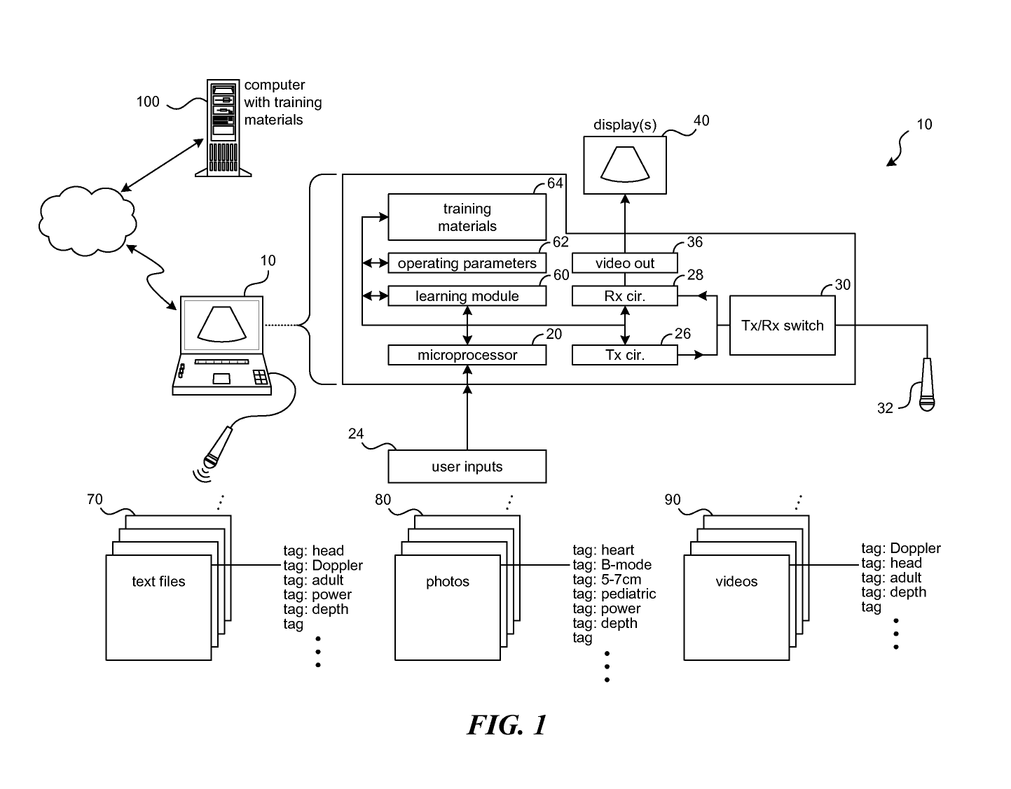

FIG. 1 illustrates an ultrasound imaging machine constructed in accordance with one embodiment of the disclosed technology. The ultrasound imaging machine can be portable or cart-based and is configured to produce ultrasonic sound waves and direct them into a body as well as to produce images from the corresponding echo signals received. The ultrasound system 10 includes one or more programmable processors 20, a set of user inputs 24 (e.g. keyboard, buttons, scroll wheel, touch pad, touch screen etc.), transmit circuitry 26 and receive circuitry 28. When transmitting ultrasound signals into the body, the transmit circuitry generates timed voltage pulses that are applied though a transmit/receive switch 30 to piezo-electric transducer elements on a probe 32. Acoustic signals received by the probe 32 are converted into corresponding electrical signals by the transducer elements. The electrical signals are routed through the transmit/receive switch 30 to the receive circuitry 28. As will be appreciated by those of ordinary skill in the art, the receive circuitry 28 includes the required amplifiers, analog to digital converters, beamformers, scan converters and other signal processing DSP's or ASICs that convert the received echo signals into still or video images using a video processor 36 for display on one or more internal or external video monitors 40. In some embodiments, a portion of the transmit/receive circuitry may be located in the probe 32 (e.g. beamforming and A/D converters). The details of the transmit and receive circuitry 26, 28 and other image processing components of the ultrasound system are considered to be known to those of ordinary skill in the art and are therefore not discussed in further detail.

In accordance with one embodiment of the disclosed technology, the ultrasound imaging system 10 includes a learning module 60, a memory 62 for storing a record of the current operating parameters of the imaging system and a memory 64 that stores a library of training materials. Such memory can include one or more of a random access memory (RAM), electronic memory chip, hard drive, solid state drive and the like. The learning module 60 is preferably implemented either as a memory storing instructions that are executable by the processor 20 or as a dedicated processor unit or ASIC that is configured to receive a user input indicating that the user would like to access a help or show me mode. In one embodiment, the learning module is built directly into the operating system of the ultrasound imaging system. In another embodiment, the learning module 60 and the memories for storing the current imaging parameters and training materials are implemented as a separate application (e.g. an “App”) that is loaded onto the ultrasound imaging system. The user activates the Learning Module App to provide the additional training functionality to the ultrasound system if the functionality is desired.

Upon detecting that the user has activated the help mode, the learning module 60 is configured to read one or more of the current operating parameters from the memory 62. Such parameters can include for example, one or more of the exam type (adult, pediatric, the region of interest etc.), the type of transducer being used, the depth of the scan, the gain applied to the received echo signals. Other parameters can include the imaging mode (B-mode, M-mode, Doppler, Power Mode etc.) From the one or more current imaging parameters that are read from the memory 62, the learning module identifies one or more training materials that are appropriate for the current imaging mode and settings of the ultrasound system. Such training materials can include text files 70, still images 80 and video/audio clips 90. In one embodiment, each of the training materials includes metadata such as tags that associate the training material with one or more imaging parameters. For example, a still image may be obtained from a pediatric kidney scan. Therefore, the tag values for the image may record B-mode, pediatric, and kidney that are useful in locating relevant training materials if the user has a question on pediatric imaging or how to perform kidney scans etc.

By reading one or more of the current imaging parameters that are stored in the memory 64, the learning module 60 is able to identify which training materials stored in memory 62 are relevant to the current operating parameters. Depending on the number of training materials that are related to the current operating parameters, the user may be shown a list of the identified training materials. Selection of any item on the list causes the corresponding training material to be presented for the user.

In some instances, the ultrasound imaging system may not have a current imaging mode because no current mode has been selected. In this case, the learning module 60 can present a list of all the training materials that are stored in the memory 64 for the user to view. As discussed above, the training materials can include textual descriptions concerning an imaging topic or a particular imaging parameter. The training materials can also include still images 80 illustrating optimal results obtainable with the ultrasound imaging system. In addition, the training materials can include audio or video clips 90 that can be selected to provide tutorials on an imaging mode or the effects of changing an imaging parameter on the quality of the images that can be produced. The video clips can also include instructions on how the probe 32 should be held or moved on the patient to perform a particular type of examination.

As imaging parameters are selected or adjusted by the user, the processor stores the imaging parameters in the memory 62. For example, if the depth of the scan is increased from 3 cm. to 7 cm., the memory 62 would be updated to store the new scan depth.

In an alternative embodiment, the imaging parameters are stored in memories (not shown) that are associated with the transmit and receive circuits 26, 28 and other circuitry of the ultrasound system. Upon detection that a user has selected the help mode, the learning module either reads the parameters from the memories or requests that the processor 20 read the parameters from the memories and return the parameter values to the learning module. Once the current operating parameters are known, they are used by the learning module to search for related training materials.

In one embodiment, the training materials are stored locally in the memory 64 of the ultrasound machine itself. In another embodiment, the training materials can be stored at a remote location 100 (e.g. a server computer run by the manufacturer of the ultrasound machine or other training provider) and recalled through a wired or wireless computer communication link. The latter embodiment can be preferable because it allows the training materials to be continually updated without having the download the new materials to the ultrasound machine. In one embodiment, the ultrasound machine determines if it has a connection to the remote computer system. If so, one or more of the current imaging parameters are sent to the remote computer to identify relevant training materials that are downloaded or streamed to the ultrasound machine. If no connection is available, then the ultrasound machine searches for relevant training materials among those that are stored locally.

In the same or an alternative embodiment, the ultrasound imaging system includes a “show me” control that can be implemented as a designated control (button, switch, knob etc.) on the ultrasound machine or as a soft key, gesture or menu item or the like on a graphical user interface or a touchscreen of the ultrasound machine. In this embodiment, training materials that can be viewed by a user are associated with an imaging technique or mode and one or more parameter settings. In one embodiment, the particular mode and parameter settings are stored as metadata tags with the training materials. The tags that are stored with the training material keep a record of such information as the type of imaging mode being discussed, the gain, the depth, the type of tissue being examined, whether the scan is for adults or children etc.

If the user would like to try and duplicate the imaging technique that is the subject of the training material being reviewed, the user selects the show me control. Upon detection of the show me control, the learning module 60 reads the imaging parameters associated with the training material. The learning module then passes the parameters to the processor that electronically provides the parameters to the transmit and receive circuitry so that the ultrasound machine is configured in the same way as the machine that is being used in the training material. In some cases, the processor may prompt the user to set some parameters or change some machine settings manually (e.g. “Please change the Adult imaging probe for a Pediatric imaging probe.” or “Please increase the gain to eleven.”). In this manner, the user can easily set the machine to use the same parameters as those described or shown in the training material. After the controls are set, the user can practice using the machine with the same imaging parameters that are used or described in the training material being reviewed. The user is then free to adjust one or more of the imaging parameters to see how the changes affect the results produced.

In the same manner described above, the training material being reviewed before the user selects the “show me” option may be stored locally on the ultrasound machine itself or streamed or downloaded from a remote location.

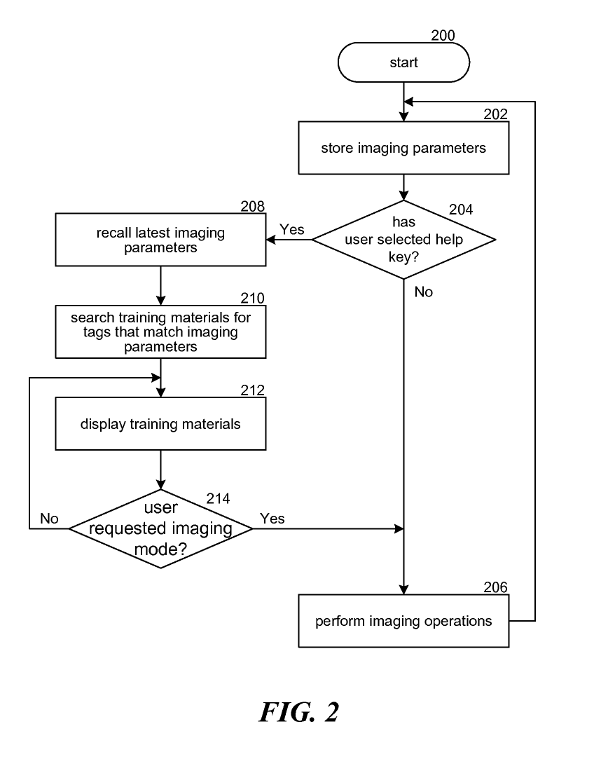

FIG. 2 shows a series of acts performed by the processor 20 and learning module 60 in accordance with embodiments of the disclosed technology in order to identify training materials that are relevant to one or more of the current operating parameters of the ultrasound imaging machine. Beginning at 200, the processor stores a record of one or more operating parameters of the ultrasound machine that are set by the operator or pre-loaded by the machine in accordance the type of examination being performed at 202. As indicated above, the parameters may be stored in the memory 62 that is dedicated to keeping a record of the imaging parameters that are being used. Alternatively, the parameters may be stored in memories that are associated with the transmit and receive circuits and other components of the ultrasound system. In yet another embodiment, a combination of memories may be used. For example, the memory 62 may store a record indicating that the current imaging mode is “Adult, cardiac” while memories associated with the transmit and receive circuits may store the particular parameter values for gain, transmit depth and other parameters to carry out adult cardiac imaging. At 204, the processor determines if the user has selected the “help me” control. If not, the ultrasound system continues in operating in the imaging mode selected at 206.

If the user has selected the help me control, the learning module 60 is invoked and one or more of the current imaging parameters of the system are determined either by reading the parameter values from the memory 62, or requesting the processor to recall parameter values that are stored in the memories that are associated with the circuits of the ultrasound system. At 210, the learning module 60 searches the available training materials for those materials having tag values that match, or are related to, the parameter values currently in use. In the current example, the learning module 60 searches the tag values for training materials related to adult and cardiac imaging. Training materials that have tag values that correspond with one or more of these parameters are displayed for the user to select. Upon selection of a particular item of training material, the material is presented for the user at 212 on a display screen or other output device that is associated with the ultrasound machine. At 214, the learning module determines if the user has requested to return to an imaging mode. If so, processing returns to step 206 and live imaging can recommence.

As discussed above, the learning module may search local copies of the training materials for those materials that have tag values matching one or more of the current operating parameters. Alternatively, the learning module may send the operating parameters to a remote processor to search a library of learning materials that are related to the current operating parameters.

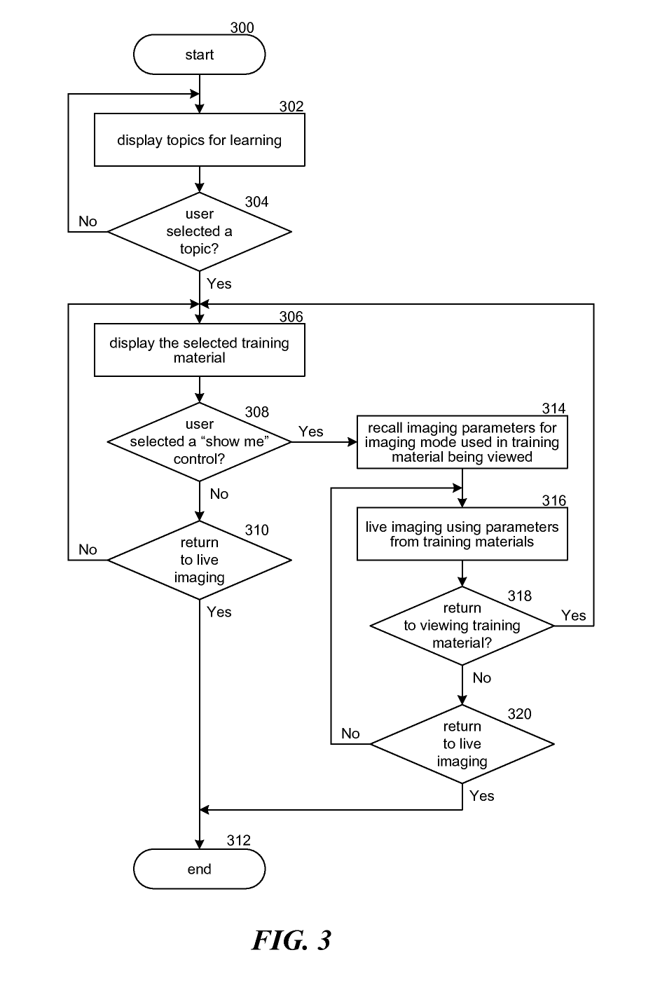

FIG. 3 show a series of steps that are performed by the learning module in addition or as an alternative to the steps shown in FIG. 2. Beginning at 300, the learning module displays a number of possible training materials for the user to review. At 304, the learning module determines if the user has selected a particular item of training material to review at 302. If so, the selected item of training material is displayed/presented for the user at 306. At 308, the learning module determines if the user has selected the “Show Me” control. If not, the learning module determines if the user has selected an option to return to live imaging at 310. If the answers at both 308 and 310 are no, then processing returns to 306 and the training materials continues to be displayed/presented for the user. If the user has requested to return to live imaging mode, then the learning module quits at 312.

If the user has selected the “Show Me” control at 308, then the learning module recalls the imaging parameters associated with the training material being reviewed at 314. The learning module provides the imaging parameters to the processor that in turn programs the transmit and receive circuitry and the other components of the imaging system with the imaging parameters being used in the training materials being reviewed. The ultrasound machine then goes into a live imaging mode at 316 so that the user can try operating the machine with the same settings described or used in the training material. At 318, the learning module determines if the user has selected to return to the reviewing the training material. If so, processing returns to step 306, where by the previously selected training material is displayed/presented again. If the answer at 318, is no, then the learning module determines if the user has selected to return to live imaging at 320. If so, the learning module quits at 312. If not, the ultrasound imaging system remains in the live imaging mode with the same parameters used in the training material being reviewed.

Although the steps shown in FIGS. 2 and 3 are described in a particular order for ease of explanation, it will be appreciated that the steps could be performed in a different order or different steps could be performed in order to achieve the functionality described.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium also can be, or can be included in, one or more separate physical components or media (e.g., multiple CDs, disks, or other storage devices). The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit).

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

To provide for interaction with a user, embodiments of the subject matter described in this specification can be implemented on an imaging system having a display device, e.g., an LCD (liquid crystal display), LED (light emitting diode), or OLED (organic light emitting diode) monitor, for displaying information to the user and a keyboard and a pointing device, e.g., a mouse or a trackball, by which the user can provide input to the computer. In some implementations, a touch screen can be used to display information and to receive input from a user. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

From the foregoing, it will be appreciated that specific embodiments of the invention have been described herein for purposes of illustration, but that various modifications may be made without deviating from the scope of the invention. Accordingly, the invention is not limited except as by the appended claims.

I/We claim:

1. An ultrasound imaging system including:
a memory configured to store one or more current operating parameters of the ultrasound imaging system;
a processor configured to execute instructions to:
determine if a user has selected a control to review an item of training material;
search a number of training materials for at least one item of training material that is related to one or more of the current operating parameters; and
present the training material that is related to one or more of the current operating parameters to the user.

2. The ultrasound imaging system of claim 1, wherein the training materials are stored with tags that represent one or more imaging parameters related to the content of the training material and wherein the processor is configured to execute instructions that search the tags of the training materials for values that are related to one or more of the current operating parameters of the ultrasound imaging system.

3. The ultrasound imaging system of claim 1, wherein the processor is configured to execute instructions that search training materials by sending one or more current operating parameters to a remote computer that stores the training materials.

4. An ultrasound imaging system including:
a memory configured to store one or more current operating parameters of the ultrasound imaging system;
a processor configured to execute instructions to:
present an item of training material to a user, wherein the training material is associated with one more operating parameters of the ultrasound system;
determine if a user has selected a control to switch to a live imaging mode;
load one or more of the operating parameters associated with the training material that is being presented to the user into circuitry of the ultrasound machine; and
display ultrasound images that are created with the parameters that were loaded into the circuitry of the ultrasound machine from the training material that is presented for the user.

5. The ultrasound imaging system of claim 4, wherein the training materials are stored with tags that represent one or more imaging parameters related to the content of the training material and wherein the processor is configured to execute instructions that read operating parameters from the tags of the training materials and program the circuitry of the ultrasound machine with one or more of the operating parameters from the tags.

6. The ultrasound imaging system of claim 4, wherein the processor is configured to execute instructions that cause the processor to receive the operating parameters stored for an item of training material from a remotely located computer.
